(19) United States
(12) Patent Application Publication
(10) Pub. No.: US 2025/0054587 A1
(43) Pub. Date: Feb. 13, 2025

(54) SYSTEMS AND METHODS FOR IDENTIFYING A HIGH-PROFILE PATIENT

(71) Applicant: Protenus, Inc., Baltimore, MD (US)

(72) Inventors: Rebecca Ellen AMBROSE, Baltimore, MD (US); Erem KAZANCIOGLU, Baltimore, MD (US); Erika DUNN-WEISS, Providence, RI (US); William STRECKFUS, Abingdon, MD (US); Nicholas CULBERTSON, Baltimore, MD (US)

(21) Appl. No.: 18/366,445

(22) Filed: Aug. 7, 2023

(51) Int. Cl. G16H 10/60; G16H 50/20; G16H 50/30

(52) U.S. Cl. CPC G16H 10/60; G16H 50/20; G16H 50/30

(57) ABSTRACT

A method includes determining a patient entity and obtaining a first set of data associated with the patient entity from one or more database systems, the first set of data including first entity identification data. The method further includes obtaining, using the first set of data, a second set of data from one or more web pages, the second set of data comprising second entity identification data and risk level indication data. The method further includes comparing the first entity identification data with the second entity identification data and determining that a threshold match exists between the first and second entity identification data. The method further includes analyzing risk level indication data against risk criteria, determining that a threshold risk exists with respect to the patient entity based on the analysis, and associating the patient entity with a high-profile indicator based upon determining that the threshold risk exists.

### TECHNICAL FIELD

The present disclosure relates generally to a computer-implemented method of identifying a high-profile status for a patient, and more specifically to comparing information from external resources to patient records to identify a patient as high-profile.

### BACKGROUND

Health service providers have an obligation to maintain privacy for patients. The risk of inappropriate accessing or breaches of patient records is greater for high-profile individuals who are well-known generally, or are subjects of a well-known incident. Therefore, greater care should be taken to prevent breaches of high-profile individuals by identifying high-profile individuals faster and more accurately.

Unless otherwise indicated herein, the materials described in this section are not prior art to the claims in this application and are not admitted to be prior art, or suggestions of the prior art, by inclusion in this section.

### SUMMARY

The following presents a simplified summary of one or more aspects in order to provide a basic understanding of such aspects. This summary is not an extensive overview of all contemplated aspects, and is intended to neither identify key or critical elements of all aspects nor delineate the scope of any or all aspects. Its sole purpose is to present some concepts of one or more aspects in a simplified form as a prelude to the more detailed description that is presented later.

In accordance with an aspect described herein a computer-implemented method for determining a high-profile status for a patient is provided. The method includes determining, by one or more processors, a patient entity, and obtaining, by the one or more processors, a first set of data associated with the patient entity from one or more database systems, the first set of data comprising first entity identification data. The method further includes obtaining, by the one or more processors and using the first set of data, a second set of data from one or more web pages, the second set of data comprising second entity identification data and risk level indication data. The method also includes comparing, by the one or more processors, the first entity identification data with the second entity identification data, and determining, by the one or more processors, that a threshold match exists between the first entity identification data and the second entity identification data based on the comparison. The method further includes analyzing, by the one or more processors, the risk level indication data against risk criteria, determining, by the one or more processors, that a threshold risk exists with respect to the patient entity based on the analysis; and associating the patient entity with a high-profile indicator based upon determining that the threshold risk exists.

In yet another aspect, a method for predicting a risk level associated with a patient entity is provided. The method includes determining, by one or more processors, one or more risk levels associated with a patient entity, the one or more risk levels including at least one of i) a first risk level determined based on first risk level indication data obtained from one or more web pages and risk criteria or ii) a second risk level provided via user input, the user input further including second risk level indication data. The method may also include storing, by the one or more processors, the one or more risk levels and the corresponding first and/or second risk level indication data in a database entry associated with the patient entity. The method also includes displaying, by the one or more processors and via a graphical user interface (GUI), the one or more risk levels and the corresponding first and/or second risk level indication data for validation, receiving, by the one or more processors and via the GUI, a feedback validating or invalidating each of the one or more risk levels; and updating, by the one or more processors, the database entry associated with the patient entity based on the feedback.

In another aspect, a system for determining a high-profile status for a patient is provided. The system includes a memory storing instructions and a trained machine learning model configured to classify patient entities into appropriate risk levels, and at least one processor operatively connected to the memory and configured to execute the instructions to perform operations. The operations may include determining a patient entity from a database system, and obtaining at least one of first risk level indication data associated with the patient entity from one or more web pages or second risk level indication data associated with the patient entity from user input. The operations may further include deriving a set of features from the at least one of the first risk level indication data or the second risk level indication data, and providing the set of features to the trained machine learning model. The operations may also include determining, using the trained machine learning model, a risk level associated with the patient entity, and associating the patient entity with a risk level indicator corresponding to the determined risk level.

To the accomplishment of the foregoing and related ends, the one or more aspects comprise the features hereinafter fully described and particularly pointed out in the claims. The following description and the annexed drawings set forth in detail certain illustrative features of the one or more aspects. These features are indicative, however, of but a few of the various ways in which the principles of various aspects may be employed, and this description is intended to include all such aspects and their equivalents.

### BRIEF DESCRIPTION OF THE DRAWINGS

The disclosed aspects will hereinafter be described in conjunction with the appended drawings, provided to illustrate and not to limit the disclosed aspects, wherein like designations denote like elements.

FIG. 1 illustrates an environment for identifying a high-profile status of a patient, in accordance with one or more embodiments of the present disclosure.

FIG. 2 is a flowchart depicting an example method for associating a patient entity with a high-profile indicator, in accordance with one or more embodiments of the present disclosure.

FIG. 3 is a flowchart depicting an example method for predicting a risk level associated with a patient entity, in accordance with one or more embodiments of the present disclosure.

FIG. 4 is a flowchart depicting an example method for determining a high-profile status for a patient using a machine learning model, in accordance with one or more embodiments of the present disclosure.

FIG. 5 illustrates an example graphical user interface (GUI) for confirming or rejecting a high-profile status determined for a patient, in accordance with one or more embodiments of the present disclosure.

FIG. 6 illustrates an example GUI for searching and viewing a list of high-profile patient entities, in accordance with one or more embodiments of the present disclosure.

FIG. 7 illustrates an example GUI for manually adding a high-profile patient entity to high-profile classification system, in accordance with one or more embodiments.

FIG. 8 illustrates an example computing device that may execute the techniques discussed herein.

### DETAILED DESCRIPTION

Aspects described herein generally relate to identification, classification, and registration of a high-profile status for a patient entity. A patient entity may be associated with a medical patient. Patients may be high-profile (e.g., a very important person, or “VIP”) due to their particular notoriety or sensitivity and risk associated with their medical records. Automated processes may be used to identify and classify high-profile patients based on information available in external resources (e.g., on the Internet) and based on information from patient medical records. High-profile patients may also be identified and classified via a user interface for maintaining custom high-profile information. Custom high-profile information maintained by a user and high-profile status determined based on external as well as internal sources may be compared, combined, and integrated as a database entity, and information related to the patient's status (e.g., high-profile or non-high-profile) may be used as inputs to train a machine learning model to automatically identify and classify high-profile patients. A resulting database entry may be viewable and editable by a user via a graphical user interface (GUI).

Medical records belonging to persons of public interest may pose an increased risk to hospitals if the records belonging to such high-profile patients are accessed inappropriately or illegally. The associated patient may also experience an increased risk as a subject of interest to the general public. Compliance and privacy personnel generally exercise heightened scrutiny toward employees who access high-profile patients' records. In some cases, specific policies and protocols may be enacted to reduce the risk of inappropriate access of high-profile patients' medical records.

It is helpful if high-profile patient records are accurately and quickly identified and classified to reduce or eliminate the number of breaches of high-profile patient records. For example, if a compliance office using conventional systems for a hospital is not aware that a patient record exists for a high-profile patient, or that a high-profile patient has recently been treated in the respective hospital, the compliance office may fail to enact specific protocols to more closely monitor access to the patient records and audit various employees' actions with heightened scrutiny and lower tolerance for anomalous behavior. In some instances, an automated classification process may be helpful to reduce risk and identify high-profile patient entities faster than a manual classification process. In some instances, a compliance officer may identify and provide context for a high-profile patient before external sources (e.g., Internet sources) identify the high-profile person as a patient in a respective hospital or institution. For example, a victim of a high-profile accident that is yet to be identified by external sources may be a target for unauthorized access. A compliance officer may be aware of the situation before any identification information is published or made available on the external sources.

Information retrieved from the Internet, or any other public/external sources (e.g., television, radio, magazines, etc.), and additional context provided by a user may be used as inputs for analytics and machine learning for future classification of patient entities into different risk levels (e.g., high-profile, non-high-profile, etc.). Patient entities may also be analyzed and classified by type and level of risk posed by possible breaches. The ability to reconcile various, sometimes conflicting, inputs may be useful to identify and reduce risk in patient privacy violations.

In one specific example, a high-profile classification system may include a series of entries in a database. Each entry in the database may include a link to a patient entity within a patient privacy monitoring system. Each entry in the database may also include links to web pages determined to be associated with the patient. Each entry may further include information from the patient's medical record identifying the patient as high-profile or belonging to a group which is considered sensitive or protected. Each entry may include information from an end user identifying the patient as high-profile, along with any specific reasons for the high-profile status. In some cases, an entry in the database may include an indication from an end user that a particular patient should not have a high-profile status. Entries in the database may also include any relevant dates, including entry creation dates, modification/update dates, and dates of medical events associated with the patient.

In another specific example, a high-profile classification system may include a user interface by which data can be viewed and edited by an end user. The high-profile classification system may include a data pipeline that takes the database entries as input and generates machine learning features that are relevant to high-profile status.

Conventionally, end users must manually identify each and every high-profile patient by flagging a patient medical record as high-profile. In some cases, there is no context for the reasons behind the high-profile status and the high-profile status may expire automatically or may need to be removed manually. These manual processes slow down identification and classification of high-profile patient records, which may lead to breaches of the patient records. The present disclosure solves this problem and/or other problems discussed above or elsewhere in the present disclosure, namely by automatically identifying, classifying, and/or registering high-profile patient records.

Because the high-profile classification system of the present disclosure enables identification, classification, and accumulation of multiple data within a database system, the high-profile system is advantageous over conventional patient medical record analysis tools. For instance, because the high-profile classification system enables classification and accumulation of multiple data from multiple sources for analysis within a system, processing power, memory, and communication resources typically needed by such a system to facilitate identifying and analyzing high-profile patient records are significantly reduced. Accordingly, the high-profile classification system results in less data transfer and data bandwidth usage for a computer/communication system. In other words, the high-profile classification system results in less required processing power and communication bandwidth in comparison to conventional systems. Additionally, in view of the foregoing, the high-profile classification system may result in a more user-friendly, consistent, reliable, accurate, and efficient method for identifying, classifying, storing, and analyzing patient records, including high-profile patient records.

Various aspects are now described with reference to the drawings. In the following description, for purposes of explanation, numerous specific details are set forth in order to provide a thorough understanding of one or more aspects. It may be evident, however, that such aspect(s) may be practiced without these specific details.

As used herein, the term “determining” or “evaluating” encompasses a wide variety of actions. For example, “determining” and “evaluating” may include calculating, computing, processing, deriving, investigating, looking up (e.g., looking up in a table, a database or other data repository, or another data structure), ascertaining, and/or the like. Also, “determining,” and “evaluating” may include receiving (e.g., receiving information), accessing (e.g., accessing data in a data repository), and/or the like. Also, “determining” may include resolving, selecting, choosing, establishing, and the like.

As used herein, the terms “element,” “module,” “component,” and “system” may refer to a computer-related entity, either hardware, a combination of hardware and software, software, or software in execution. For example, a module may be, but is not limited to being, a machine-executable process running on a processor, a processor, an object, a thread of execution, a machine-executable program, and/or a computer. By way of illustration, both a process running on a server and the server may be a module or a component. One or more modules or components may reside within a process and/or thread of execution. In some implementations, a module may be localized on one computer and/or distributed among two or more computers.

Various aspects or features will be presented herein in terms of systems that may include a number of devices, components, modules, and the like. It is to be understood and appreciated that the various systems may include additional devices, components, modules, etc. and/or may not include all of the devices, components, modules, etc., discussed in connection with the figures. A combination of these approaches may also be used.

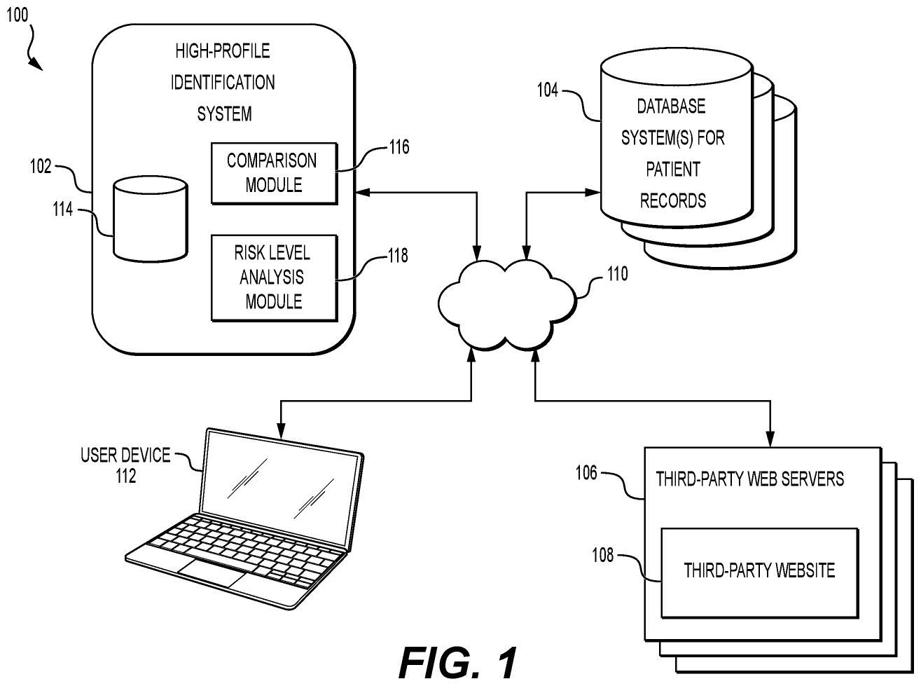

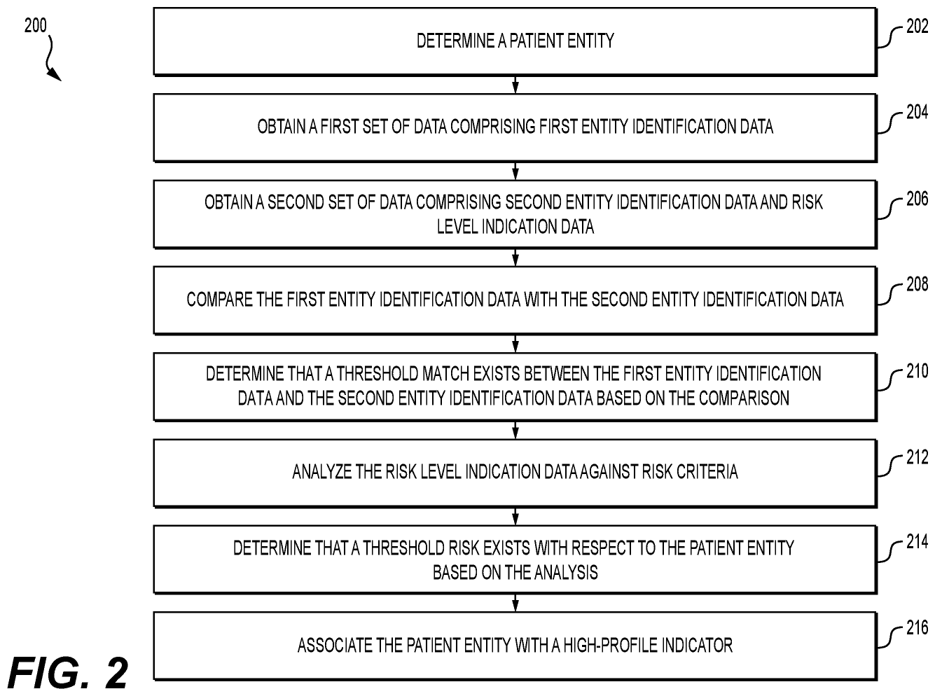

Referring to FIGS. 1-8, aspects are depicted with reference to one or more components and one or more methods that may perform the actions or functions described herein. Although the operations described below in FIGS. 2-4 are presented in a particular order and/or as being performed by an example component, it should be understood that the ordering of the actions and the components performing the actions may be varied, depending on the implementation. Moreover, it should be understood that the following actions or functions may be performed by a specially-programmed processor, a processor executing specially-programmed software or computer-readable media, or by any other combination of a hardware component and/or a software component capable of performing the described actions or functions.

FIG. 1 is a diagram illustrating an environment 100 for identifying a high-profile status of a patient entity, according to one or more embodiments of the present disclosure. A high-profile classification system 102 communicates with one or more other components of environment 100 across a network 110, including a user device 112, one or more database system(s) 104 for patient records, and one or more web servers 106. The high-profile classification system 102 is connected via network 110 to database system(s) 104 and may also be connected with user device 112 and web server(s) 106. Each component of environment 100 may be located remotely from each other.

Database system(s) 104 for patient records may include one or more databases containing patient records including electronic medical records (EMRs) or electronic health records (EHRs). While not shown in FIG. 1, environment 100 may include additional database systems that each contain patient records. The database system(s) 104 may be associated with a healthcare service provider, which may include a hospital, hospital system, clinic, or medical group. The data related to the patient and stored in the one or more database systems 104 may include, by way of example, demographics, medical history, medication and allergies, immunization status, laboratory test results, radiology images, vital signs, personal statistics like age and weight, and billing information. Database system(s) 104 may also include activity and access logs related to the patient records. For example, employees of the healthcare service provider may access the patient records using one or more applications and these accesses may be logged and stored in association with the patient records. By way of example, the data related to an employee user and stored in the one or more database system(s) 104 may include access authorization data (e.g., login information, cryptographic keys, lists of systems the user has access to, access and activity logs, etc.), demographic and personal information, or associated users (e.g., managers or direct reports whom the user manages). Database system(s) 104 may include servers that perform computations and processes related to storage and access of patient medical records or employee user records.

By way of example, an employee of a healthcare service provider may access database system(s) 104 via user device 112. Healthcare service providers may use the various systems, as supported by the database system(s) 104, to perform administrative functions, track patient records, medical history, and medical visits, store patient information, book and remind patients of upcoming visits, interact with patients online, diagnose medical conditions, generate and transmit electronic prescriptions, automate daily operations, and manage billing, documentation, and inventory. An employee may also be a compliance officer that ensures that patient privacy is maintained and risks of breach are reduced. Determining possible anomalous activity and/or inappropriate accessing (also referred to herein as “breach”) of the data contained in database system(s) 104 may be a manual or an automated process based on analysis of the data. Data may be collected from database system(s) 104 and analyzed to determine possible breaches. For example, the collected data can include electronic patient data (e.g., from an EMR) and data related to accessing the electronic patient data (e.g., an identifier of an employee accessing the EMR data, a time of accessing the EMR data, etc.). The data may also include human resources (HR) data that can indicate information related to the employees accessing the electronic patient data. The collected data may be analyzed to detect whether one or more accesses of the data may possibly be a breach of the data. Analysis may differ (e.g., greater scrutiny, lower tolerance for anomalous activity) depending on whether the patient entity potentially breached is classified as high-profile. If a possible breach is detected, one or more alerts may be generated (e.g., to one or more interfaces) for further investigation as to whether the access is inappropriate given additional context around the access. Moreover, though EMR data is generally referred to herein, the concepts described can be applied to substantially any electronically stored patient data.

Third-party web servers 106 may host one or more third-party websites 108. Each third-party web server 106 may include one or more third-party electronic devices (e.g., computing devices or groups of computing devices), such as individual servers or groups of servers operating in a distributed manner. A third-party web server 106 may communicate with high-profile classification system 102 or user device 112 via network 110. For example, third-party web servers 106 may receive requests for data from user device 112 or high-profile classification system 102. Third party web servers 106 may send the requested data via the network 110 to the user device 112 or high-profile classification system 102. In some examples, a user may interact with third-party web servers via user device 112. To facilitate the interaction with the user, the third-party web servers 106 may present one or more third-party websites 108 to user device 112. In some embodiments, third-party web servers 106 may host third-party websites created and administered by news organizations and other public sources. While the term “website” is used herein, the third-party website 108 may be a user interface other than a website or a webpage, such as a user interface of a client-side application executing on the user device 112, such as the user interface of an app executing on a laptop or a smartphone of the user and communicating with the third party web servers 106. The third-party web servers may regularly send updates (e.g., news updates) to high-profile classification system 102.

High-profile classification system 102 may include one or more modules, such as comparison module 116 and risk level analysis module 118, and a database system 114 for storage and management of patient records that have been classified as high-profile. High-profile classification system 102 may receive regular updates (e.g., news updates and information) from third-party web servers 106 to determine whether a patient entity stored in database system 104 should be labeled as “high-profile.” In some embodiments, high-profile classification system 102 regularly scrapes (e.g., extracts content and data from) the one or more third-party websites 108 hosted by third-party web servers 106. High-profile classification system 102 may include in database system 114 a list of websites to regularly retrieve for data extraction. The list of websites may include local news websites, social media websites, or any other website that provides public information (e.g., any information readily, easily, and legally accessible to the public). High-profile classification system 102 may analyze the extracted data from third-party web servers 106 to identify names and other identification data for people identified in a third-party website 108 as being possibly popular, important, hospitalized, injured, ill, or otherwise involved in a medical emergency. In some cases, a machine learning model may be employed to identify identification data for people identified in the third-party website 108.

Database system 114 may include patient entities that have been classified as high-profile. Database system 114 may also allow for removal or modification of high-profile patient entities. High-profile classification system 102 may manage and maintain the database system 114 which may include entries of high-profile patient entities or other entities that include indicators of non-high-profile status. Each entry may include entity metadata such as creation, modification, and expiration dates.

In some cases, a high-profile database entry within the database system 114 may include the entirety of the associated patient entity. In other cases, the high-profile database entry within database system 114 may include one or more links to, pointers to, or reference numbers for any associated patient records (e.g., patient records stores in database systems 104) that have been identified and associated with a high-profile indicator. The high-profile database entry may include one or more links to one or more patient privacy monitoring database systems (not shown), including database entries within the one or more patient privacy monitoring database systems. The high-profile database entry may include links to the web pages from which data were obtained. In some instances, the high-profile database entry may include the source code (e.g., HTML code) for the web pages such that the webpage may be displayed within a GUI provided by the high-profile classification system 102. The high-profile database entry may also include raw data and text or images obtained from the web pages, which may be displayed with a user interface for an end user. The high-profile database entry may include a confidence rate at which the identification threshold match and threshold risk was determined. The high-profile database entry may also include any information from the patient record(s) that may have been used to classify the patient entity as high-profile (e.g., a flag, an alias, redacted information, etc.).

Comparison module 116 may compare names and other identification data with identification data from patient entities stored in database system(s) 104. Comparison module 116 may determine, using one or more thresholds, that a match exists between the web page identification data and the patient entity identification data.

Upon determination that a person identified in data extracted from one or more websites is the same as a person identified in a patient entity, risk level analysis module 118 may analyze a risk level based on risk level indication data also extracted from the one or more web pages. If risk level analysis module 118 determines that a risk exists with respect to the patient entity, high-profile classification system 102 may associate the patient entity with a high-profile indicator.

The user device 112 is configured to enable a user to access database system 104. In some examples, the user may be an employee of a healthcare service provider, such as a physician, physician's assistant (PA), nurse, nurse practitioner, receptionist, certified nursing assistant (CNA), medial assistant, hospital administrator, compliance officer, or anybody else who may have a reason to access a patient record. The user may use the user device 112 to record or access information related to a patient stored in database system 104. The user device 112 is a computer system such as, for example, a desktop computer, a laptop computer, a tablet, a smart cellular phone, a smart watch, or other wearable computer, etc. The user device 112 includes one or more applications, e.g., a program, plugin, browser extension, etc., installed on a memory of the user device 112. The applications may include one or more of system control software, system monitoring software, software development tools, etc.

In some embodiments, at least one of the applications is associated with and configured to communicate with one or more of the other components in the environment 100, such as database system 104 or high-profile classification system 102. For example, the at least one application may be executed on the user device 112 to communicate with database systems 104.

The network 110 over which the one or more components of the environment 100 communicate includes one or more wired and/or wireless networks, such as a wide area network (“WAN”), a local area network (“LAN”), personal area network (“PAN”), a cellular network (e.g., a 3G network, a 4G network, a 5G network, etc.) or the like. In some embodiments, the network 110 includes the Internet, and information and data provided between various systems occurs online. “Online” means connecting to or accessing source data or information from a location remote from other devices or networks coupled to the internet. Alternatively, “online” refers to connecting or accessing a network (wired or wireless) via a mobile communications network or device. The user device 112, high-profile classification system 102, and database system 104 are connected via the network 110, using one or more standard communication protocols. The user device 112, the high-profile classification system 102, and the database system 104 transmit and receive communications from each other across the network 110.

Although depicted as separate components in FIG. 1, it should be understood that a component or portion of a component in the environment 100 is, in some embodiments, integrated with or incorporated into one or more other components. As one example, high-profile classification system 102 and database systems 104 may be integrated into a single component of environment 100. In some embodiments, operations or aspects of one or more of the components discussed above are distributed amongst one or more other components. Any suitable arrangement and/or integration of the various systems and devices of the environment 100 may be used.

In the following disclosure, various acts are described as performed or executed by a component from FIG. 1, such as the user device 112 or the high-profile classification system 102, or components thereof. However, it should be understood that in various aspects, various components of the environment 100 discussed above execute instructions or perform acts including the acts discussed below. An act performed by a device is considered to be performed by a processor, actuator, or the like associated with that device. Further, it should be understood that in various embodiments, various steps can be added, omitted, and/or rearranged in any suitable manner.

FIG. 2 is a flowchart depicting an example method 200 for associating a patient entity with a high-profile indicator, according to one or more embodiments. Method 200 may be performed by one or more components of the high-profile classification system 102 of FIG. 1, according to some embodiments of the disclosure.

At step 202, the method 200 includes determining a patient entity. A patient entity may be a data object that represents a patient at a hospital or medical clinic. A patient entity may include one or more medical records associated with a particular person. In some embodiments, the medical records associated with the patient may be stored in one or more database system(s) 104.

At step 204, the method 200 includes obtaining a first set of data associated with the patient entity from one or more database systems. High-profile classification system 102 may query (e.g., send a request to) the one or more database systems to obtain first entity identification data (e.g., identification data stored in the one or more database systems in association with the patient entity). The first entity identification data may include, for example, a person's name, date of birth, age, gender, ethnicity, location, address, phone number, etc.

At step 206, the method 200 includes obtaining a second set of data from one or more external sources, such as, e.g., web pages. The first set of data obtained at step 204 associated with a patient may be used to obtain the second set of data from the one or more web pages. The second set of data obtained at step 206 may include second entity identification data and risk level indication data, both of which are extracted from the one or more web pages. The one or more web pages may be examples of individual web pages from third-party websites 108 hosted on third-party web servers 106 of FIG. 1. The one or more web pages may include web pages from more than one website.

The second entity identification data may include names, dates, locations, events, etc., which may be similar and/or comparable to the types of data included in the first entity identification data. Each of the first entity identification data and the second entity identification data may also include a hospital name, a hospital location, or a type of medical event (e.g., an injury, disease, infection, stroke, heart attack, etc.). In some embodiments, the second entity identification data may be extracted from web pages using a machine learning model that has been trained to identify names, dates, locations, and events from one or more blocks of text. Data may also be extracted from one or more images of the one or more webpages, in some cases using a trained machine learning model. In addition to receiving regular updates from one or more websites, the high-profile classification system 102 may scrape multiple websites on a regular basis (e.g., weekly, daily, hourly, multiple times per hour) to obtain data related to potential high-profile patients.

Risk level indication data may include any information that may pertain to the patient entity and is indicative of the patient's high-profile status. For example, risk level indication data may include an individual's occupation, criminal history, job history, the number of websites identifying the patient, incidents related to the patient, amount of time elapsed since a website identified the patient, length and content of the website, etc. Risk level indication data may be used for risk level determination, which is discussed in more detail below.

Certain items may be identified as relevant when obtaining identification and risk level indication data from web pages such as names of high-profile individuals (e.g., celebrities, politicians, very important persons (“VIPs”), “famous” persons, musical artists, movie stars, notable persons, etc.). The high-profile classification system 102 may maintain a list of high-profile individuals by regularly scraping web pages that maintain lists of celebrities and well-known or famous people. Celebrities who are hospitalized or involved in some kind of accident may be the subject of one or more news stories related to the hospitalization and/or accident. The high-profile classification system 102 may obtain data from news websites related to the hospitalization and/or accident including a celebrity's name, the time, date, and location of the accident, and the celebrity's gender and age if included in the web page. The high-profile classification system 102 also obtains data from web pages including information about possible high-profile accidents or injuries. For example, an individual in Florida may have been injured by an alligator and has been potentially hospitalized. The story is on a news website that high-profile classification system 102 regularly monitors and scrapes. Identification and risk level indication data obtained in this specific example may include the individual's name (if the name appears in the story), the individual's gender, the individual's age, the location of the injury, and a name of a hospital where the individual was hospitalized. Furthermore, the high-profile classification system 102 may determine that an accident is a high-profile accident if more than a threshold number of web pages has a story related to the accident or if a web page with details of the accident is shared on social media.

At step 208, the method 200 includes comparing the first entity identification data with the second entity identification data obtained from the one or more web pages. Comparing may include determining which data points correspond to each other between the first entity identification data and the second entity identification data. For example, the high-profile classification system 102 may determine that at least one data point from the web pages corresponds to an individual's name and therefore should be compared to the name of the patient associated with the patient entity.

At step 210, the method 200 includes determining that a threshold match exists between the first entity identification data and the second entity identification data from the web page(s) based on the comparison of step 208. The threshold match may be based on a set of threshold matching criteria that may be predetermined. In some embodiments, the set of threshold matching criteria may evolve over time as the system learns how to better match records using a pre-trained machine learning model or by updating the machine learning model with new data. The set of threshold criteria may include a number of one or more types of data points of demographic information that may be used to determine a threshold match at a particular confidence rate.

By way of example, the set of threshold criteria may include two points of demographic information such as a name and age. In this instance, the data points from the web pages may include a name and an age of an individual. If the name and age from the data points from the web pages match the name and age of a patient entity stored in the healthcare service provider's database system(s), a threshold match may be determined to exist, even if no other demographic information match between the web page data points and the patient entity demographic information. The confidence rate for this set of threshold criteria may be lower than the confidence rate for a set of threshold criteria that includes additional demographic information such as gender, ethnicity, location, etc. In other words, a set of threshold criteria that includes more points of demographic information may have a higher confidence rate. However, each type of demographic information used for a threshold criteria may be assigned a weight for the confidence rate. For example, name and age may have a greater weight for confidence rate than gender or ethnicity because names and ages may be more unique and are less likely to be shared among multiple people than gender and ethnicity, which may be shared by a large amount of people. However, the overall confidence rate may be increased by including gender and ethnicity in addition to name and age in the set of threshold criteria, due to the possibility that a person shares a name and age with another, but is of a different gender or ethnicity. For example, it is possible that a patient has the same name and age as an individual identified in a local news story about a high-profile accident, but the patient is male and the high-profile accident victim is female. If the threshold criteria only included name and age, the high-profile classification system 102 may incorrectly classify the patient as high-profile. But if the threshold criteria included the gender, the high-profile classification system 102 would correctly differentiate between the two, and would not improperly classify the patient entity associated with the patient as high-profile.

High-profile classification system 102 may be configured to account for mistakes in the data, including misspellings of names and slight differences in ages. In other words, patient entities or internet sources that are associated with the same individual may include minor mistakes as a result of data entry or modification, and a threshold match may still exist even with these minor mistakes.

At step 212, the method 200 includes analyzing the risk level indication data against risk criteria. The risk criteria may define one or more data types indicative of i) a high risk level in case one or more records associated with an individual are exposed or compromised, and/or ii) popularity or notoriety associated with an individual. Various factors that may cause harm are assessed to determine how likely those factors lead to negative consequences. In some cases, risk criteria may include a list of patient occupations that may increase a risk of misappropriation or breach of patient records. Risk criteria may also include a criminal history data or notoriety within a community. Particular incidents or accidents may be more prone to risk for inappropriate accessing of patient records involved in those incidents or accidents. Such incidents or accidents may be included as risk criteria. Further risk criteria may include a number of websites (e.g., articles) that an individual appears in, a time since a website mentioning the individual was published, a length or content of the relevant website, etc.

At step 214, the method 200 includes determining that a threshold risk exists with respect to the patient entity based on the analysis of step 212. If the risk level indication data indicates that, based on the risk criteria, there is a very low risk or no risk at all of breach and/or implications thereof, the method 200 may end and take no further action.

There may be different thresholds, classes, or levels of risk criteria which may allow for different tiers of risk or high-profile status (e.g., a first, second, or third tier). For example, some risk level indication data may indicate a very high risk based on the fame or notoriety of the patient. In contrast, some risk may be low, for example, a victim of a bicycle accident reported on the news may involve some risk that the patient's information may be breached or used inappropriately, but the risk may be lower than for a well-known celebrity. In the case of the bicycle accident victim, the high-profile classification system 102 may assign a lower risk tier which may result in lesser restrictions on the patient entity. Further, patient entities with a high-profile status may include an expiration date for the high-profile status. In some cases, a lower risk tier patient entity may have a sooner expiration date such that the patient entity is not considered high-profile for as long as a well-known celebrity who might be assigned a higher-risk tier.

At step 216, upon determining that there is a threshold match between the first entity identification data and the second entity identification data, and upon determining that a threshold risk exists with respect to the patient entity, the method 200 includes associating the patient entity with a high-profile indicator. Associating the patient entity with a high-profile indicator may also be referred to herein “classifying,” “registering,” or “flagging” a patient entity as high-profile. The high-profile indicator may classify the patient associated with the patient entity as a person of public interest, in accordance with one or more embodiments.

Associating the patient entity with the high-profile indicator may include creating a new database entry or updating an existing database entry in database systems 104 or 114. The new or updated database entry may include at least a portion of the first set of data and at least a portion of the second set of data obtained from the one or more web pages. The new or updated database entry may also include a reason for associating the patient entity with the high-profile indicator and/or database entry creation, expiration, and/or modification dates.

In some embodiments, associating the patient with the high-profile indicator includes transmitting a data object to database systems 104, wherein the data object includes instructions to flag the patient entity as high-profile. The indicator may also include information such as links to web pages, a confidence rate at which the threshold match was determined, and any additional information that may be useful for an end user to determine whether the determination as high-profile is correct.

In some embodiments, the method 200 further includes, upon associating the patient entity with the high-profile indicator, restricting access to the one or more records associated with the patient entity. Restricting access to the patient entity may include restricting which employees of the healthcare service provider has access to view or edit any of the information of the high-profile patient entity. In some cases, employees may only view or edit the high-profile patient entity if the employee has a particular access privilege or level, which may be determined by a compliance department of the healthcare service provider. In some cases, restricting access to the patient entity may include redacting or restricting which demographic information or medical records of the high-profile patient may be viewed or edited by some or all employees of the healthcare service provider.

In some embodiments, the method 200 further includes auditing access events to one or more records associated with the patient entity based on a lower tolerance for anomalous activity than a standard tolerance for anomalous activity used for patient entities that are not associated with a high-profile indicator. Due to the higher sensitivity and risk for breach of privacy, high-profile patient entities may be monitored and audited with a lower tolerance than is normally afforded for patient entities (e.g., high-profile patient entities may be monitored at a different/higher suspicion threshold). For example, following auditing or monitoring of user activity in relation to patient records, a suspicion score may be generated by a machine learning model regarding user access to particular patient entities. A threshold to determine suspicious or anomalous activity may be lower (e.g., require less suspicious or anomalous activity) for high-profile patient entities than for non-high-profile patient entities. Anomalous activity may include illegal or unauthorized access of one or more records associated with the patient entity (e.g., by employees of the healthcare service provider). Anomalous activity may also refer to drug diversion activity.

A manual override process may be incorporated into method 200 which allows for external (e.g., manual) identification and classification of a patient entity that should be identified as high-profile, regardless of whether a threshold risk exists. Additionally, a manual override process may be incorporated which allows for external identification and classification of patient entities associated with a high-profile indicator, but should not be associated with a high-profile indicator. In one or more embodiments, a graphical user interface (GUI) may be displayed on a computing device of a user for manually updating the patient entity or the high-profile patient entity. The GUI may enable a user to perform the manual override process discussed herein by enabling the user to select patient entities that should or should not be associated with a high-profile indicator. The classification of patient entities that should or should not be identified as high-profile may be recorded by the high-profile classification system 102 as a “rule,” such that the rule remains applicable during subsequent operations. The high-profile classification system 102 may also allow users to modify or delete rules previously set during a manual override process, via one or more graphical elements of the GUI. The GUI may further enable a user to add a new entry to the high-profile database system (e.g., high-profile database system 114) using one or more graphical elements of the GUI. The GUI may also enable a user to remove a patient entity from the high-profile database system using one or more graphical elements of the GUI. In some embodiments, the graphical elements used to add a patient entity to the high-profile database system may be the same or may be different than the graphical elements used to remove a database entity from the second database system.

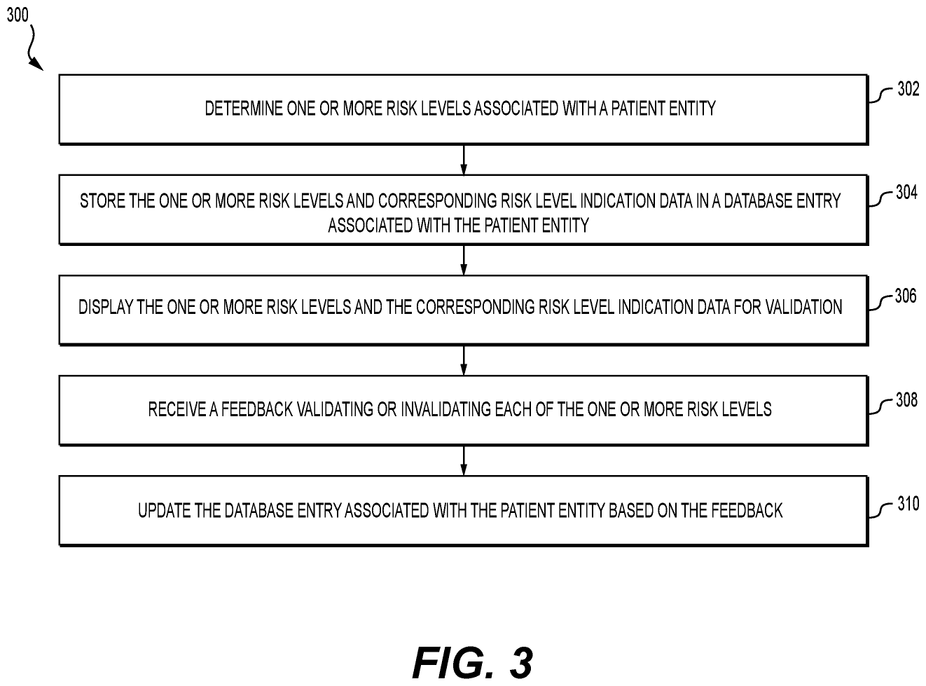

FIG. 3 is a flowchart depicting an example method 300 for predicting a risk level associated with a patient entity, according to one or more embodiments. Method 300 may be performed by one or more components of the high-profile classification system 102 of FIG. 1, according to some embodiments of the disclosure.

At step 302, the method 300 includes determining one or more risk levels associated with a patient entity. The one or more risk levels may include a first risk level determined based on first risk level indication data obtained from one or more web pages and risk criteria. The risk levels may also include a second risk level provided via user input, which user input may include additional and/or different second risk level indication data, which may include reasons as to why the second risk level should be associated with the patient entity. The first risk level based on first risk level indication data obtained from one or more web pages and risk criteria may be determined in accordance with techniques discussed in the present disclosure. For example, the first risk level based on first risk level indication data obtained from one or more web pages and risk criteria may be determined by analyzing the first risk level indication data against the risk criteria and determining that a threshold risk exists with respect to the patient entity based on the analysis. Further, the patient entity may be associated with the first risk level based upon determining that the threshold risk exists.

In some cases, the user input may indicate a particular risk level (e.g., that the patient should receive a high-profile status). For example, the patient entity may be associated with a flag that indicates whether an end user (e.g., compliance officer) believes a patient should receive high-profile status. As another example, the patient entity may be associated with obfuscated demographic data such as an alias (e.g., the patient's real name does not appear in the observable sections of the patient entity). The obfuscated data may be a result of user input. As a further example, certain portions of data associated with the patient entity may have been redacted such that certain information is only observable/accessible by employees with sufficient access clearance. Additionally, access to a patient entity may be manually restricted, which may be indicative of a particular risk level.

At step 304, the method 300 includes storing the one or more risk levels and the corresponding risk level indication data in a database entry associated with the patient entity. The data may be stored in database system 114 or database systems 104.

At step 306, the method 300 includes displaying, via a GUI, the one or more risk levels and the corresponding risk level indication data for validation. The patient entity associated with the high-profile indicator and at least a portion of the risk level indication data may be displayed using the GUI. Using the GUI, the patient entity associated with the high-profile indicator and the one or more web pages may be displayed to a user. In some cases, only extracted identification data may be displayed instead of the one or more web pages. The GUI may include a prompt for the user to confirm or reject the classification of the patient entity as high-profile. The GUI display may include all the identification data and risk level indication data related to the patient entity and web pages.

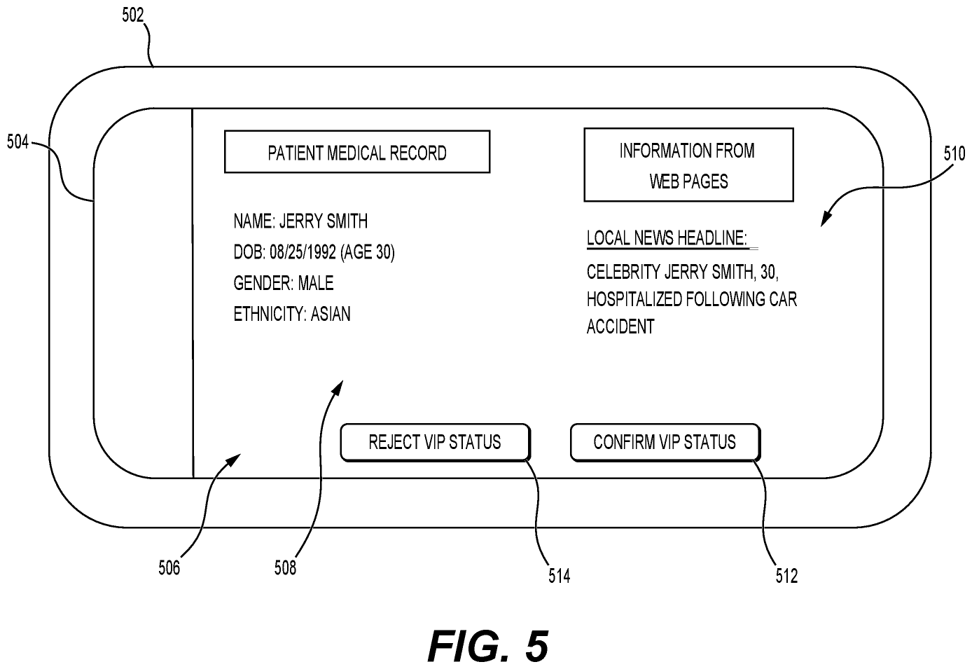

At step 308, the method 300 includes receiving feedback, via the GUI, validating or invalidating each of the one or more risk levels. For example, a user may, via the GUI, provide feedback indicating whether the patient entity is correctly associated with the high-profile indicator. A representative GUI is illustrated in FIG. 5. The user may provide an indication, via the GUI and in response to the prompt, that the high-profile classification was correct or not. In some embodiments, the user may set a time period after which the high-profile classification will expire. The user may set the high-profile classification to expire immediately effectively rejecting a high-profile status for the patient entity.

At step 310, the method 300 includes updating the database entry associated with the patient entity based on the feedback. If the feedback indicates that the risk levels or the high-profile classification are incorrect, the high-profile classification system 102 may remove the high-profile indicator association or be prevented from associating the patient entity with the high-profile indicator temporarily or permanently. If the feedback indicates that the risk levels or the high-profile classification are correct, the high-profile classification system 102 may continue to associate the patient entity with the high-profile indicator.

A set of features may be derived from the first and/or second risk level identification data corresponding to the one or more risk levels and used, along with the feedback as the ground truth label, to train a machine learning model configured to classify patient entities into appropriate risk levels. As used herein, a “machine learning model” generally encompasses instructions, data, and/or a model configured to receive input, and apply one or more of a weight, bias, classification, or analysis on the input to generate an output. The output may include, for example, a classification of the input, an analysis based on the input, a design, process, prediction, or recommendation associated with the input, or any other suitable type of output. A machine learning model is generally trained using training data, e.g., experiential data and/or samples of input data, which are fed into the model in order to establish, tune, or modify one or more aspects of the model, e.g., the weights, biases, criteria for forming classifications or clusters, or the like. Aspects of a machine learning model may operate on an input linearly, in parallel, via a network (e.g., a neural network), or via any suitable configuration.

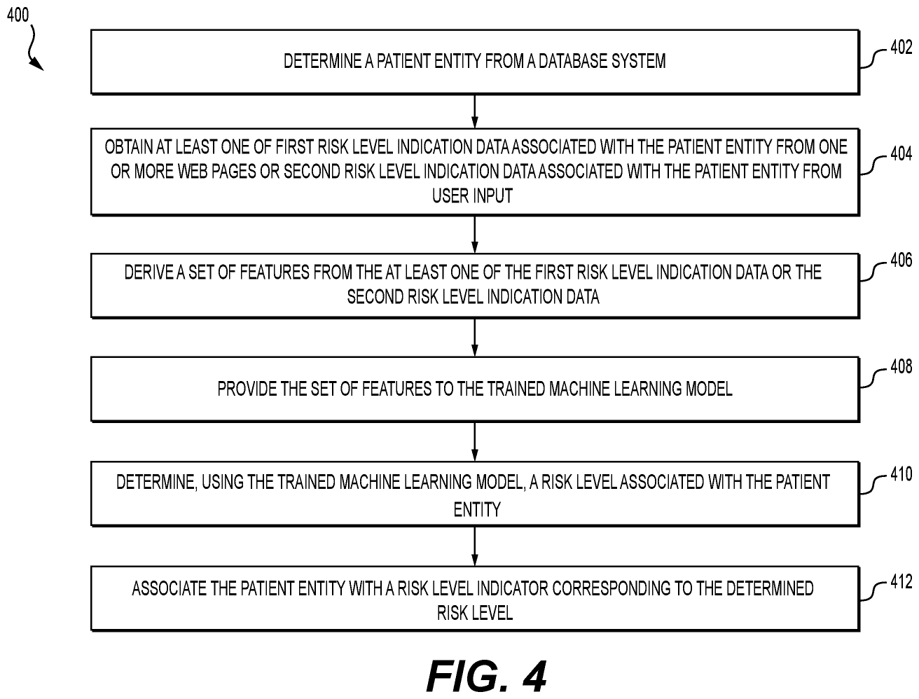

FIG. 4 is a flowchart depicting an example method 400 for determining a high-profile status for a patient using a machine learning model, in accordance with one or more embodiments of the present disclosure. Method 400 may be performed by one or more components of the high-profile classification system 102 of FIG. 1, according to some embodiments of the disclosure.

At step 402, the method 400 includes determining a patient entity from a database system. In some embodiments, the database system may be the database systems 104 and determining a patient may include receiving one or more patient records which are combined into a patient entity.

At step 404, the method 400 includes obtaining at least one of a first risk level indication data associated with the patient entity from one or more web pages or second risk level indication associated with the patient entity from user input. This may be accomplished in accordance with various techniques discussed in the present disclosure.

At step 406, the method 400 includes deriving a set of features from the at least one of the first risk level indication data or the second risk level indication data. As one example, the set of features may include demographic identification data points. Certain features may also correspond to the types of data defined by the risk criteria, which may be used to determine risk levels associated with breach of private patient information. The set of features may further describe a quantity and a quality of one or more types of input including web-based input (e.g., a website and its contents), manual user input (e.g., compliance officer input including free text description of a high-profile reason), and information derived from a medical record associated with an individual (e.g., a high-profile status flag/indicator). For example, a patient mentioned in a news article on a website may be associated with features such as a number of additional articles that the patient appears in, an amount of time elapsed since the publication of the articles, the length or content of the articles, etc.

At step 408, the method 400 includes providing the set of features to a trained machine learning model. The set of features may be inputs to the trained machine learning model that has been trained previously using the same or similar features to determine risk levels for patient entities.

At step 410, the method 400 includes determining, using the trained machine learning model, a risk level associated with the patient entity. The risk level may correspond to the implications of the patient data being breached or compromised. The risk level may be binary (e.g., high-profile status or non-high-profile status) or there may be different tiers of risk levels, as discussed above in the present disclosure. At step 412, the method 400 includes associating the patient entity with a risk level indicator corresponding to the risk level determined at step 410.

FIGS. 5-7 illustrate a user interface including features of the high-profile classification system 102, according to one or more embodiments of the present disclosure. The components of the high-profile classification system 102 may provide, along with and/or in combination with other components, one or more GUIs. In particular, the components may allow a user to interact with a collection of display elements for a variety of purposes. For instance, FIGS. 5-7 and the description that follows illustrate various example embodiments of the user interfaces and corresponding features.

For example, FIG. 5 illustrates a user device 502 that may implement one or more components or features of the high-profile classification system 102. For purposes of the present disclosure, the user device 502 may be an example of user device 112 of FIG. 1. As shown in FIG. 5, in some embodiments, the user device 502 is a handheld device, such as a tablet device. As used herein, the term “handheld device” refers to a device sized and configured to be held/operated in one or more hands of a user. In additional or alternative examples, however, any other suitable computing device, such as, but not limited to, a mobile phone device, larger wireless device, laptop or desktop computer, a personal digital assistant device, and/or any other suitable computing device may perform one or more of the processes and/or operations described herein.

In some embodiments, the user device 502 includes a touch screen display 504 that can display user interfaces. Furthermore, the user device 502 receives and/or detects user input via the touch screen display 504. As used herein, a “touch screen display” refers to the display of a touch screen device. In one or more embodiments, a touch screen device may be the user device 502 with at least one surface upon which a user may perform touch gestures (e.g., a laptop, a tablet computer, a personal digital assistant, a media player, a mobile phone, etc.). Additionally, or alternatively, the user device 502 may include any other suitable input device, such as a touch pad, mouse, or keyboard.

As shown in FIG. 5, a display of the user device 502 displays a high-profile classification system GUI 506 provided by the high-profile classification system 102, which may be accessible by the user device 502. For example, as discussed above with reference to FIG. 1, the user device 502 (e.g., user device 112) may access the high-profile classification system 102 and/or the database system 104 via a network (e.g., network 110). GUI 506 may include a patient record section 508, a web page information section 510, and confirmation buttons 512, 514. In one embodiment, GUI 506 may be configured to be displayed via an Internet browser installed on the user device 502, in response to an appropriate address (e.g., URL) provided to the browser or via a browser extension configured to communicate with the high-profile classification system 102 for displaying the GUI 506 via the browser.

Patient record section 508 may display one or more medical records associated with the patient entity that has been classified as high-profile by high-profile classification system 102, as discussed above with respect to FIG. 3. In some cases, only the most relevant (e.g., matching) demographic information is displayed in patient record window 508 for user reference. In some embodiments, a user may be able to interact with patient record window 508 to expand and display additional information regarding the patient medical record. Any user input that indicates that the patient is high-profile may be highlighted.

Web page information section 510 may display one or more web pages, or relevant data extracted from such web pages, that resulted in the classification of the patient entity as high-profile. The web pages may be displayed as they would be displayed in an internet browser. In some embodiments, only relevant information may be displayed as text or images. Furthermore, matching components of the data may be highlighted to illustrate why the high-profile classification system 102 determined that the patient entity should be associated with the high-profile indicator.

If a user (e.g., compliance officer) is satisfied that the patient medical record matches the information from the one or more web pages, and that the risk level is appropriate given the circumstances and context, the user may confirm the high-profile (“VIP”) status by selecting “Confirm VIP Status” button 512. If user is not satisfied, either because the patient medical record does not match the information from the web pages, or because the risk level is not appropriate, then the user may reject the high-profile status by selecting “Reject VIP Status” button 514. In some embodiments not shown in FIG. 5, a user may be presented with a patient entity that has been identified as high-profile, and an option to set a time period after which the high-profile status will expire. If the user sets the expiration date as the current date, the user may effectively reject the high-profile status for the particular patient entity going forward.

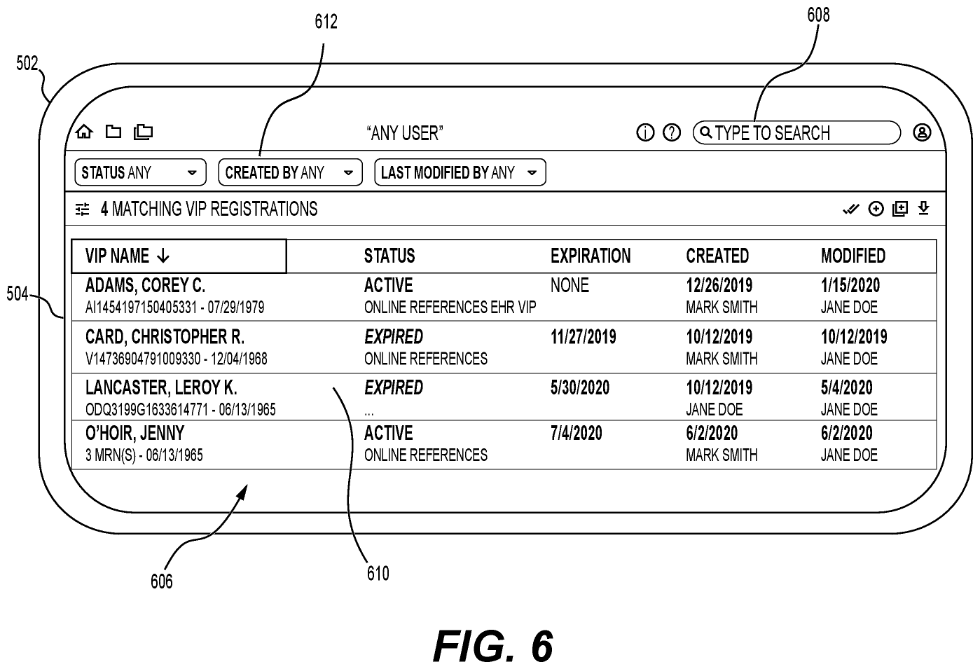

Referring to FIG. 6, GUI 606 may include search bar 608, an entity list 610, and one or more filters 612. Search bar 608 may receive text from a user to search the list of high-profile database entries stored in the database system 114 of high-profile classification system 102. Upon entering search text, the entity list 610 may change to show which high-profile patient entities match the searched text. Generally, names may be searched, but other data points of information or demographic information may be searched including dates of birth (age), other relevant dates (e.g., dates of creation, modification, etc.), and other demographic information may be searched. In some embodiments, database entries of patients that are non-high-profile may also be searched and retrieved via GUI 606 in a similar manner.

Entity list 610 may show a list of all patient entities that have been identified as high-profile, along with relevant dates and a current high-profile status. As discussed above, patient entities that are non-high-profile may also be listed responsive to a corresponding search query. Entity list 610 may display the patient entities that match search query as inputted in the search bar 608. In some cases, the displayed list of patient entities may be narrowed by using one or more filters 612. For example, listed high-profile patient entities may include those that have expired and are no longer considered “high-profile.” These entities may be retained in the database system but are marked as “expired.” A user may restrict the search results, and thus the displayed list of entities in the entity list 610 by filtering out any expired high-profile patient entities. Entity list 610 may display the date of expiration, the date of creation, and/or the date of any modification of each of the high-profile patient entities.

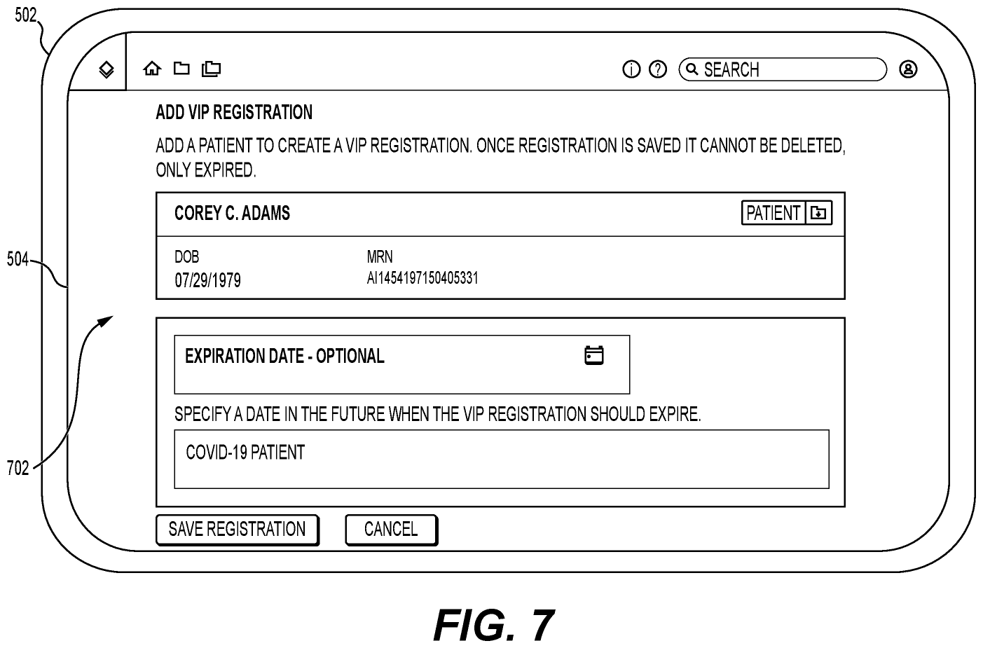

Referring to FIG. 7, the high-profile classification system 102 displays a high-profile database entity creation page 702. Entity creation page 702 allows a user to manually create a new high-profile database entity. The user may search and add a patient entity that should be identified as a high-profile patient entity. The user may enter an expiration date for the high-profile status or may choose to leave the status indefinitely. The user may also add comments indicating context and reasons for identifying the particular patient entity as a high-profile patient entity. These comments will remain a part of the high-profile patient entity for future review and possible revision. In some embodiments, a high-profile database entity that is created may not be deleted, but may only be expired.

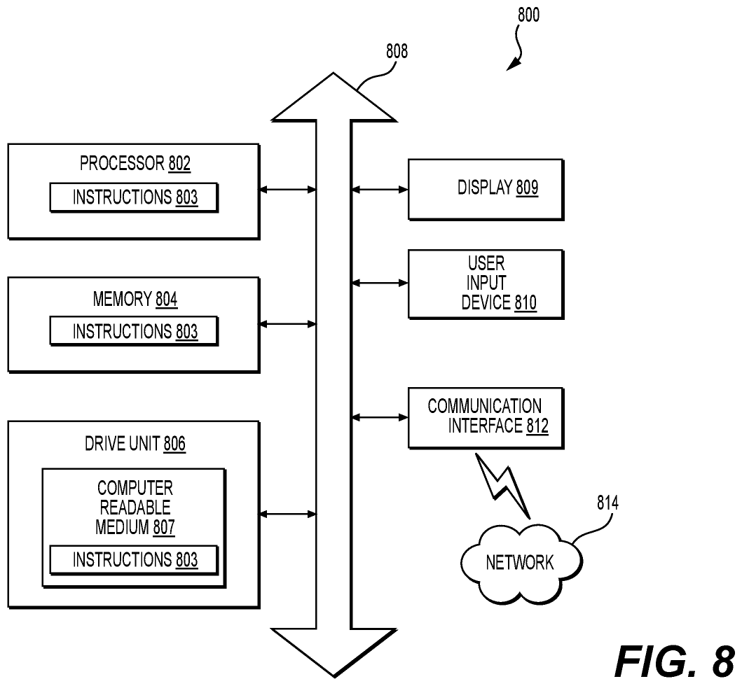

FIG. 8 is a block diagram illustrating various example system components for use in accordance with the disclosed systems and methods. The computer system 800 includes a processor 802, e.g., a central processing unit (CPU), a graphics processing unit (GPU), or both. The processor 802 is a component in a variety of systems. For example, the processor 802 is part of a standard personal computer or a workstation. The processor 802 is one or more processors, digital signal processors, application specific integrated circuits, field programmable gate arrays, servers, networks, digital circuits, analog circuits, combinations thereof, or other now known or later developed devices for analyzing and processing data. The processor 802 implements a software program, such as code generated manually (i.e., programmed).

The computer system 800 includes a memory 804 that communicates via bus 808. Memory 804 is a main memory, a static memory, or a dynamic memory. Memory 804 includes, but is not limited to computer-readable storage media such as various types of volatile and non-volatile storage media, including but not limited to random access memory, read-only memory, programmable read-only memory, electrically programmable read-only memory, electrically erasable read-only memory, flash memory, magnetic tape or disk, optical media and the like. In one implementation, the memory 804 includes a cache or random-access memory for the processor 802. In alternative implementations, the memory 804 is separate from the processor 802, such as a cache memory of a processor, the system memory, or other memory. Memory 804 is an external storage device or database for storing data. Examples include a hard drive, compact disc (“CD”), digital video disc (“DVD”), memory card, memory stick, floppy disc, universal serial bus (“USB”) memory device, or any other device operative to store data. The memory 804 is operable to store instructions executable by the processor 802. The functions, acts, or tasks illustrated in the figures or described herein are performed by processor 802 executing the instructions stored in memory 804. The functions, acts, or tasks are independent of the particular type of instruction set, storage media, processor, or processing strategy and are performed by software, hardware, integrated circuits, firmware, micro-code, and the like, operating alone or in combination. Likewise, processing strategies include multiprocessing, multitasking, parallel processing, and the like.

As shown, the computer system 800 further includes a display 809, such as a liquid crystal display (LCD), an organic light emitting diode (OLED), a flat panel display, a solid-state display, a cathode ray tube (CRT), a projector, a printer or other now known or later developed display device for outputting determined information. The display 809 acts as an interface for the user to see the functioning of the processor 802, or specifically as an interface with the software stored in the memory 804 or in the drive unit 806.

Additionally or alternatively, the computer system 800 includes an input/output device 810 configured to allow a user to interact with any of the components of the computer system 800. The input/output device 810 is a number pad, a keyboard, a cursor control device, such as a mouse, a joystick, touch screen display, remote control, or any other device operative to interact with the computer system 800.

The computer system 800 also includes the drive unit 806 implemented as a disk or optical drive. The drive unit 806 includes a computer-readable medium 807 in which one or more sets of instructions 803, e.g. software, is embedded. Further, the sets of instructions 803 embodies one or more of the methods or logic as described herein. Instructions 803 resides completely or partially within memory 804 and/or within processor 802 during execution by the computer system 800. The memory 804 and the processor 802 also include computer-readable media as discussed above.

In some systems, computer-readable medium 807 includes the set of instructions 803 or receives and executes the set of instructions 803 responsive to a propagated signal so that a device connected to network 814 communicates voice, video, audio, images, or any other data over network 814. Further, the sets of instructions 803 are transmitted or received over the network 814 via the communication port or interface 812, and/or using the bus 808. The communication port or interface 812 is a part of the processor 802 or is a separate component. The communication port or interface 812 is created in software or is a physical connection in hardware. The communication port or interface 812 is configured to connect with the network 814, external media, display 809, or any other components in the computer system 800, or combinations thereof. The connection with network 814 is a physical connection, such as a wired Ethernet connection, or is established wirelessly as discussed below. Likewise, the additional connections with other components of the computer system 800 are physical connections or are established wirelessly. Network 814 alternatively be directly connected to the bus 808.

While the computer-readable medium 807 is shown to be a single medium, the term “computer-readable medium” includes a single medium or multiple media, such as a centralized or distributed database, and/or associated caches and servers that store one or more sets of instructions. The term “computer-readable medium” also includes any medium that is capable of storing, encoding, or carrying a set of instructions for execution by a processor or that causes a computer system to perform any one or more of the methods or operations disclosed herein. The computer-readable medium 807 is non-transitory, and may be tangible.

The computer-readable medium 807 includes a solid-state memory such as a memory card or other package that houses one or more non-volatile read-only memories. The computer-readable medium 807 is a random-access memory or other volatile re-writable memory. Additionally or alternatively, the computer-readable medium 807 includes a magneto-optical or optical medium, such as a disk or tapes or other storage device to capture carrier wave signals such as a signal communicated over a transmission medium. A digital file attachment to an e-mail or other self-contained information archive or set of archives is considered a distribution medium that is a tangible storage medium. Accordingly, the disclosure is considered to include any one or more of a computer-readable medium or a distribution medium and other equivalents and successor media, in which data or instructions are stored.

In an alternative implementation, dedicated hardware implementations, such as application specific integrated circuits, programmable logic arrays, and other hardware devices, is constructed to implement one or more of the methods described herein. Applications that include the apparatus and systems of various implementations broadly include a variety of electronic and computer systems. One or more implementations described herein implement functions using two or more specific interconnected hardware modules or devices with related control and data signals that are communicated between and through the modules, or as portions of an application-specific integrated circuit. Accordingly, the present system encompasses software, firmware, and hardware implementations.

Computer system 800 is connected to network 814. Network 814 defines one or more networks including wired or wireless networks. The wireless network is a cellular telephone network, an 802.11, 802.16, 802.20, or WiMAX network. Further, such networks include a public network, such as the Internet, a private network, such as an intranet, or combinations thereof, and utilizes a variety of networking protocols now available or later developed including, but not limited to TCP/IP based networking protocols. Network 814 includes wide area networks (WAN), such as the Internet, local area networks (LAN), campus area networks, metropolitan area networks, a direct connection such as through a Universal Serial Bus (USB) port, or any other networks that allows for data communication. Network 814 is configured to couple one computing device to another computing device to enable communication of data between the devices. Network 814 is generally enabled to employ any form of machine-readable media for communicating information from one device to another. Network 814 includes communication methods by which information travels between computing devices. Network 814 is divided into sub-networks. The sub-networks allow access to all of the other components connected thereto or the sub-networks restrict access between the components. Network 814 is regarded as a public or private network connection and includes, for example, a virtual private network or an encryption or other security mechanism employed over the public Internet, or the like.

While the foregoing has been described in conjunction with the example aspects outlined above and further described in the figures, various alternatives, modifications, variations, improvements, and/or substantial equivalents, whether known or that are or may be presently unforeseen, may become apparent to those having at least ordinary skill in the art. Accordingly, the exemplary aspects, as set forth above, are intended to be illustrative, not limiting. Various changes may be made without departing from the spirit and scope of the subject matter described herein. Therefore, aspects described herein intended to embrace all known or later-developed alternatives, modifications, variations, improvements, and/or substantial equivalents.

1. A computer-implemented method for determining a high-profile status for a patient, comprising:
determining, by one or more processors, a patient entity;
obtaining, by the one or more processors, a first set of data associated with the patient entity from one or more database systems, the first set of data comprising first entity identification data;
obtaining, by the one or more processors and using the first set of data, a second set of data from one or more web pages, the second set of data comprising second entity identification data and risk level indication data;
comparing, by the one or more processors, the first entity identification data with the second entity identification data;
determining, by the one or more processors, that a threshold match exists between the first entity identification data and the second entity identification data based on the comparison;
executing, by the one or more processors, a trained machine learning model by inputting the risk level indication data and/or risk criteria to generate a risk level associated with the patient entity, wherein
the machine learning model has been trained by:
inputting, to the machine learning model, first risk level indication training data obtained from one or more web pages and risk criteria and/or second risk level indication training data provided via user input;
analyzing the first risk level indication training data and/or the second risk level indication training data to assign weights or biases to one or more factors identified in the first risk level indication training data and/or the second risk level indication training data, and
determining an association between the one or more risk levels and the one or more factors based on the weights or biases assigned to the one or more factors;
determining, by the one or more processors, that a threshold risk exists with respect to the patient entity based on the generated risk level;
associating, by the one or more processors, the patient entity with a high-profile indicator based upon determining that the threshold risk exists; and
upon determining that the threshold risk exists with respect to the patient entity, restricting, by the one or more processors, network access to the first identification data associated with the patient entity.

2. The method of claim 1, wherein each of the first entity identification data and the second entity identification data comprises at least one of: a name, an age, an ethnicity, or a location.

3. The method of claim 1, wherein the risk level indication data comprises at least one of: occupational data, criminal data, or incident data.

4. The method of claim 1, wherein the risk criteria define one or more data types indicative of a high risk level in case one or more records associated with the patient entity are exposed or compromised.

5. The method of claim 1, further comprising:
displaying, by the one or more processors and via a graphical user interface (GUI), the patient entity associated with the high-profile indicator and at least a portion of the risk level indication data; and
receiving, by the one or more processors and via the GUI, a feedback indicating whether the patient entity is correctly associated with the high-profile indicator.

6. The method of claim 5, further comprising:
further training, by the one or more processors and using the feedback indicating whether the patient entity is correctly associated with the high-profile indicator and a set of features derived from the risk level identification data, the machine learning model configured to classify patient entities into appropriate risk levels.

7. The method of claim 1, wherein associating the patient entity with the high-profile indicator comprises at least one of:
creating a new database entry in the one or more database systems or another database system; or
updating an existing database entry in the one or more database systems or another database system.

8. The method of claim 7, wherein the new database entry or the updated existing database entry includes at least one of:
at least a portion of the second set of data obtained from the one or more web pages;
at least a portion of the first set of data obtained from the one or more database systems;
a reason for associating the patient entity with the high-profile indicator; or
database entity creation, expiration, and/or modification dates.

9. The method of claim 1, wherein the high-profile indicator identifies the patient as a person of public interest in the one or more database systems.

10. The method of claim 1, wherein associating the patient entity with the high-profile indicator comprises transmitting a data object to the one or more database systems, the data object including instructions to flag the patient entity as high-profile.

11. The method of claim 1, further comprising restricting access to one or more records associated with the patient entity upon associating the patient with the high-profile indicator.

12. The method of claim 1, further comprising:
auditing, by the one or more processors, access events to one or more records associated with the patient entity based on a lower tolerance for anomalous activity than a standard tolerance for anomalous activity used for patient entities that are not associated with a high-profile indicator.

13. The method of claim 12, wherein the anomalous activity comprises at least one of:
illegal access to the one or more records associated with the patient entity;
unauthorized access to the one or more records associated with the patient entity; or
drug diversion activity.

14. The method of claim 1, wherein each of the first entity identification data and the second entity identification data comprises at least one of: a hospital name, a hospital location, or a type of medical event.

15. The method of claim 1, wherein the patient entity is associated with a plurality of medical records pertaining to a single patient.

16. A computer-implemented method of predicting a risk level associated with a patient entity, comprising:
determining, by one or more processors and using a machine learning model, one or more risk levels associated with a patient entity, wherein the machine learning model has been trained by i) inputting, to the machine learning model, first risk level indication training data obtained from one or more web pages and risk criteria and/or second risk level indication training data provided via user input, ii) analyzing the first risk level indication training data and/or the second risk level indication training data to assign weights or biases to one or more factors identified in the first risk level indication training data and/or the second risk level indication training data, and iii) determining an association between the one or more risk levels and the one or more factors based on the weights or biases assigned to the one or more factors, the one or more risk levels including at least one of i) a first risk level determined based on first risk level indication data or ii) a second risk level provided via user input, the user input further including second risk level indication data;
storing, by the one or more processors, the one or more risk levels and corresponding first and/or second risk level indication data in a database entry associated with the patient entity;
displaying, by the one or more processors and via a graphical user interface (GUI), the one or more risk levels and the corresponding first and/or second risk level indication data for validation;
receiving, by the one or more processors and via the GUI, a feedback validating or invalidating each of the one or more risk levels;
updating, by the one or more processors, the database entry associated with the patient entity based on the feedback;
updating, by the one or more processors and using the feedback and a set of features derived from the first and/or second risk level indication data corresponding to the one or more risk levels, the machine learning model configured to classify the patient entity into an appropriate risk level;
analyzing risk level indication data for the patient entity against the risk criteria;
determining that a threshold risk exists with respect to the patient entity based on the analysis;
associating the patient entity with an elevated risk level based upon determining that the threshold risk exists; and
upon determining that the threshold risk exists, restricting, by the one or more processors, network access to identification data associated with the patient entity.

17. (canceled)

18. (canceled)

19. The method of claim 16, wherein the second risk level indication data indicates why the second risk level should be associated with the patient entity.

20. A system for determining a high-profile status for a patient, comprising:
a memory storing instructions and a trained machine learning model configured to classify patient entities into appropriate risk levels; and
at least one processor operatively connected to the memory and configured to execute the instructions to perform operations including:
determining a patient entity from a database system;
obtaining at least one of first risk level indication data associated with the patient entity from one or more web pages and second risk level indication data associated with the patient entity from user input, the second risk level indication data including a risk level;
deriving a set of features from the first risk level indication data and the second risk level indication data;
providing the set of features to the trained machine learning model;
determining, using the trained machine learning model, an updated risk level associated with the patient entity, wherein
the machine learning model has been trained by:
inputting, to the machine learning model, first risk level indication training data obtained from one or more web pages and risk criteria and/or second risk level indication training data provided via user input;
analyzing the first risk level indication training data and/or the second risk level indication training data to assign weights or biases to one or more factors identified in the first risk level indication training data and/or the second risk level indication training data, and
determining an association between the one or more risk levels and the one or more factors based on the weights or biases assigned to the one or more factors; and
associating the patient entity with a risk level indicator corresponding to the determined updated risk level; and
upon determining that the updated risk level exceeds a threshold risk level, associating the patient entity with a high-profile status and restricting, by the at least one processor, network access to identification data associated with the patient entity.
